(19) United States
(12) Patent Application Publication
(10) Pub. No.: US 2020/0338023 A1
(43) Pub. Date: Oct. 29, 2020

(54) NON-TOXIC TOPICAL FORMULATIONS OF DAPSONE

(71) Applicant: IMUNEKS FARMA ILAC SANAYI VE TICARET ANONIM SIRKETI, ISTANBUL (TR)

(72) Inventors: MEHMET NEVZAT PISAK, ISTANBUL (TR); SEVAL KORKMAZ, ISTANBUL (TR)

(21) Appl. No.: 16/426,784

(22) Filed: May 30, 2019

(30) Foreign Application Priority Data
Apr. 24, 2019 (TR) 2019/06066

(51) Int. Cl. A61K 31/145; A61K 47/10; A61K 47/34; A61K 47/38; A61K 9/00

(52) U.S. Cl. CPC A61K 31/145; A61K 47/10; A61K 9/0014; A61K 47/38; A61K 47/34

(57) ABSTRACT

The present invention relates to a topical composition comprising dapsone as active agent, at least one emollient and at least one solubilizing agent. The present invention provides a topical formulation of dapsone which is easily applicable to the acne lesion with required dose.

### CROSS REFERENCE TO RELATED APPLICATION

This application claims priority to Turkish Patent Application No. 2019/06066 filed Apr. 24, 2019, which is hereby incorporated by reference in its entirety.

### TECHNICAL FIELD

The present invention relates to a topical composition of dapsone comprising at least one solubilizing agent and at least one emollient.

### BACKGROUND ART

Acne is a group of common skin conditions which can be caused by bacteria or can also be marked by inflammation. Classification and diagnosis of acne conditions can be contradictory and the treatment can vary from patient to patient Therefore, there is a lot of compositions for treatment of acne available in the prior art.

Some of the currently available products include antibiotics (topical and systemic), benzoyl peroxide, retinoids (topical and systemic), dapsone, and a number of other compounds. Dapsone (4,4′-diaminodiphenyl sulfone) is a medicament for use in the treatment of skin diseases characterized by the abnormal infiltration of neutrophils, such as Dermatitis herpetiformis, linear IgA dermatosis, pustular psoriasis, pyoderma gangrenosum, acne vulgaris, and Sweet's Syndrome. The anti-acne molecule dapsone is marketed as a commercial product Aczone®.

U.S. Pat. No. 9,161,926B2 discloses a topical formulation of dapsone comprising %30 to %40 of diethylene monoethyl ether (Transcutol®) with 2 to about 6% by weight of a polymeric viscosity builder consisting of acrylamide/sodium acryloyldimethyl taurate copolymer. It has been disclosed in prior art that the use of a polymeric viscosity builder; acrylamide/sodium acryloyldimethyl taurate copolymer minimizes the intensity of yellowing of the composition. In addition, the polymeric viscosity builder influences dapsone crystallization. But, acrylamide was shown to be a neurotoxicant, reproductive toxicant, and carcinogen in animal species. It has also shown to be an initiator for skin tumors in mice. (Toxicology Data Network)

EP2459172A1 discloses dermatological composition comprising dapsone and adapalene formulated with 1.5% w/w benzyl alcohol, transcutol, 5-25% w/w PEG 400, 0.01% w/w EDTA, and 0.03% w/w citric acid. U.S. Pat. No. 5,863,560A discloses a dermatological gel composition of dapsone including carbomer, ethoxydiglycol and methyl paraben. All of the above prior arts disclosed topical formulations of dapsone comprising parabens as preservatives.

Parabens are esters of p-hydroxybenzoic acid that are used as antimicrobial preservatives in cosmetics, food packaging, and pharmaceuticals. Parabens are often used in topical compositions and numerous articles were published related to the risks of parabens exposure and it's link to certain types of cancers activation and development. Thus, the avoidance of paraben exposure is beneficial especially for a topical treatment for acne which would require prolonged exposure to the formulation due to the chronic nature of the disease, as it would be known by one with ordinary skill in the art

While the safety of topical formulation is provided, it is also important to have a topical formulation with maximum applicability to the site of the acne lesions. There is still a need in the art for a topical formulation of dapsone which is homogeneous, stable and easily applicable to the acne lesion with required dose and which also provide an increased patient compliance through increasing the ease of use.

### BRIEF DESCRIPTION OF THE DRAWINGS

FIG. 1 is a photograph of samples of the formulations from examples 1, 2, 3, 4, 5, 6 and 7 following storage for 1 month at 20-25° C. at 60% relative humidity.

### DETAILED DESCRIPTION OF THE INVENTION

The present invention relates to a topical composition comprising dapsone as active agent, at least one glycerine based emollient and at least one solubilizing agent

In one embodiment of the present invention, the topical composition comprises between 3 to 10% of dapsone and preferably between 5 to 8% of dapsone by weight of the total unit dosage form. Most preferably, dapsone is found at a concentration of 3%, 5% or 7.5% by weight of the total unit dosage form.

In one embodiment of the present invention, the emollient is selected from the group consisting of glycerin, Macrogol, glycerol ricinoleate (Kolliphor® ELP), caprylic/capric triglyceride (Myritol® 318), PEG glyceryl fatty acid esters such as PEG-8 glyceryl caprylate/caprate (Labrasol®); PEG-4 glyceryl caprylate/caprate (Labrafac Hydro® WL 1219); PEG-32 glyceryl laurate (Gelucire® 44/14); PEG-6 glyceryl mono oleate (Labrafil® M 1944 CS); PEG-6 glyceryl linoleate (Labrafil® M 2125 CS); glycerol triacetate; monoglycerides and acetylated monoglycerides; glycerol monodicocoate (e.g. Imwitor® 928); glycerol monocaprylate (e.g. Imwitor® 308); mono-and di-acetylated monoglycerides; Polyglyceryl-3 dioleate (Plurol® Oleique CC 497) and their combinations.

In one preferred embodiment of the present invention, the emollient is selected from the group consisting of glycerin, PEG-8 glyceryl caprylate/caprate, Polyglyceryl-3 dioleate, PEG-4 glyceryl caprylate/caprate, PEG-32 glyceryl laurate, PEG-6 glyceryl mono oleate, PEG-6 glyceryl linoleate, Macrogolglycerol ricinoleate, and their combinations; and more preferably glycerin and/or PEG-8 glyceryl caprylate/caprate.

PEG-8 glyceryl caprylate/caprate or Caprylocaproyl Macrogol-8 Glycerides (Labrasol®) is a saturated polyglycolized glyceride consisting of mono-, di- and triglycerides and with mono- and di-fatty acids of polyethylene glycol (PEG).

In one embodiment of the present invention, the pharmaceutical composition comprises between 1 to 40% of at least one emollient; preferably between 2 to 20% of at least one emollient and more preferably between 5 to 20% of at least one emollient by weight of the total unit dosage form.

The solubilizing agent is selected from the group consisting of Butylene glycol, Polyethylene glycols (PEG), e.g. PEG 400, Diethylene glycol monoethyl ether (Transcutol®), Tetrahydrofurfuryl PEG ether (Tetraglycol or Glycofurol), N-methyl pyrrolidone (Pharmasolve), 2-Pyrrolidone (Soluphor P), short chain alcohols (e.g. ethanol, benzyl alcohol, isopropanol, phenethyl alcohol) and their combinations. The solubilizing agent is preferably selected from the group consisting of diethylene glycol monoethyl ether, glycerol, N-methyl pyrrolidone, benzyl alcohol and their combinations. The solubilizing agent is more preferably diethylene glycol monoethyl ether and/or benzyl alcohol.

In one embodiment of the present invention, the pharmaceutical composition comprises between 10 to 40% of a solubilizing agent, preferably between 15 to 40% of a solubilizing agent by weight of the total unit dosage form.

According to the present invention, the pharmaceutical composition comprises dapsone, at least one glycerine based emollient and at least one solubilizing agent wherein the weight ratio of solubilizing agent to emollient is between 10:1 and 1:1.

It has been surprisingly found that the use of a glycerin based emollient together with a solubilizing agent disrupts the crystalline phases which can be formed within the formulation and decreases the crystal size of dapsone within the formulation whilst minimizing the discoloration of the composition. In addition to providing a good aesthetic profile without discoloration, the use of a glycerin based emollient together with a solubilizing agent, also increases the solubility of dapsone.

In one embodiment, the topical composition according to the present invention does not comprise paraben. The necessity of using paraben as preservative is resolved by using BHT or a short chain alcohol which can be act as a preservative in the composition according to the present invention.

The preservative according to the present invention can be Butylhydroxy toluene(BHT) and/or a short chain alcohol selected from ethanol, benzyl alcohol, isopropanol, phenethyl alcohol and the like.

In one embodiment, the topical composition according to the present invention does not comprise an acylamide or acrylamide based excipients for any function.

In one embodiment of the present invention, the topical composition of dapsone comprises at least one solubilizing agent together with at least one emollient, wherein the preservative is a short chain alcohol. Herein, short chain alcohols act both as a preservative and a co-surfactant

Thus, it simplifies the manufacturing process and the formulation and also the stability of the formulation is provided.

In one embodiment, the pharmaceutical composition comprises between 1 to 5% of short chain alcohol by weight of the total unit dosage form.

In one embodiment of the present invention, the pharmaceutical composition comprises butylated hydroxytoluene (BHT). BHT function as a preservative and antioxidant within the formulation, thus it would also simplify the process of manufacturing and make it more cost effective. The composition comprises between 0.02 to 0.5% of BHT by weight of the total unit dosage form.

In one preferred embodiment of the present invention, the topical composition of dapsone comprises at least one glycerin based emollient and the combination of diethylene glycol monoethyl ether with a short chain alcohol as solubilizing agent or BHT as preservative. Thus, use of an unsafe preservative such as paraben is avoided and a soluble uniform topical formulation is provided.

In one embodiment, the topical composition of dapsone comprises a solubilizing agent and a glycerin based emollient, with a preservative.

In another embodiment, the topical composition of dapsone comprises the solubilizing agent diethylene glycol monoethyl ether and the emollient glycerine.

In another embodiment, the topical composition of dapsone comprises the solubilizing agent diethylene glycol monoethyl ether and the emollient PEG-8 glyceryl caprylate/caprate.

In yet another preferred embodiment, the topical composition of dapsone comprises the solubilizing agent diethylene glycol monoethyl ether and the emollient glycerine.

It has been observed that the addition of at least another emollient to the composition of the present invention has created a more aesthetic composition with less grittiness. The second emollient other than the glycerin based emollient can be selected as polysorbate, preferably polysorbate 20 and/or squalene.

In one embodiment, the topical composition of dapsone comprises a solubilizer, a glycerine based emollient, and a second emollient with a preservative.

In yet another preferred embodiment, the topical composition of dapsone comprises the solubilizing agent of diethylene glycol monoethyl ether and the emollients of glycerine and/or squalene.

In another embodiment of the present invention, the topical composition further comprises at least one neutralizing agent Said neutralizing agent is selected from sodium hydroxide or triethanolamine and preferably triethanolamine. In addition, the topical composition comprises between 0.05 to 0.4% of neutralizing agent by weight of the total unit dosage form.

In one embodiment of the present invention, the pharmaceutical composition further comprises at least one gelling agent The gelling agent, especially when selected as a cellulose derivate also had a positive result as far as the particle size was concerned. It has been surprisingly observed that the use of a gelling agent, along with a glycerin based emollient, provided improved aesthetics, no discoloration, and spreadability of the dapsone topical formulation. In a preferred embodiment the weight ratio of the gelling agent to dapsone is between 1:5 and 1:1.

The composition comprises between 0.5 to 8% of a gelling agent and preferably between 1 to 7% of gelling agent by weight of the total unit dosage form. The amount of gelling agent is calibrated to these ranges in order to provide a topical composition with better spreadability.

In one preferred embodiment of the present invention, the gelling agent is selected from cellulose derivatives, preferably from the group consisting of carboxymethyl cellulose, ethylcellulose, hydroxyethyl cellulose, hydroxypropyl cellulose, methylcellulose and the like and their combinations. Additionally, the use of a cellulose derivative as the preferred gelling agent of the composition can also decrease the mean particle size of dapsone within the formulation when used at appropriate weight ratios in relation to dapsone. Furthermore, cellulose derivatives are known to be extremely safe for use and non-toxic.

In one embodiment, the mean particle size of dapsone crystals in the topical composition according to the present invention is between 10 to 50 μm, preferably 15 to 30 μm. As used herein, the term “mean particle size” refers to an average of the crystal particle diameters of all the particles in a population of the particles.

In one embodiment of the present invention, the topical composition may be gel, emulsion, cream, liquid, paste or lotion and preferably a gel.

In one embodiment of the present invention, the topical composition of dapsone is used in the treatment of acne, leprosy and skin condition known as dermatitis herpetiformis.

In one embodiment, the present invention provides a method for obtaining said topical composition comprises the following steps:
a) solubilizing dapsone with the solubilizing agent,
b) water and preservative is added to the dapsone solution in phase a),
c) the cellulose derivative is dispersed into the glycerine based emollient with an optional second or third emollient.
d) The cellulose phase is added to dapsone solution obtained with the solubilizer, preservative and water.
e) The mixture is left to gel.

According to the present invention, the method for obtaining the topical composition comprises the step of mixing dapsone with a dispersion wherein a cellulose derivative is dispersed into the glycerine based emollient.

Unless otherwise defined, all technical and scientific terms used herein have the same meaning as commonly understood by one of ordinary skill in the art In addition, the materials, methods, and examples are illustrative only and not intended to be limiting.

The invention now will be described in particularity with the following illustrative examples; however, the scope of the present invention is not intended to be, and shall not be, limited to the exemplified embodiments below.

### EXAMPLES

### Example 1

| Ingredients | Functions | % w/w |
|---|---|---|
| Dapsone | Active agent | 5.00 |
| Diethylene glycol monoethylether | Solubilizer | 20.00 |
| (Transutol ® P, Gattefoose) - | | |
| Hydroxyethyl cellulose(HEC) | Cellulose | 2.00 |
| (Natrosol ® 250 HHX Pharma | derivative | |
| (Ashland)) - | | |
| Capryl-caproyl macrogol-8 | Glycerin based | 10.00 |
| glyceride (Acconon ® MC8-2 | emollient | |
| EP/NF (Abitec)) - | | |
| Butylhydroxy toluene (BHT) - | Preservative | 0.05 |
| Triethanolamine - | Neutralizing | 0.2 |
| | agent | |
| Demineralized Water | | 62.75 |

### Manufacturing Process

1. Dapsone is solubilized into Diethylene glycol monoethyl ether.
2. Add BHT and water to dapsone solution while stirring at 350 rpm for 15 minutes and obtain phase with dapsone
3. Hydroxyethylcellulose is dispersed into capryl-caproyl macrogol-8 glyceride while stirring at approximately 350 rpm for at least 15 minutes.
4. Add triethanolamine and stir 10 minutes more and obtain HEC phase.
5. The phase with dapsone is then added to the HEC phase under stirring at 250 rpm and mixed for 3 hours.
6. The mixture is then left for 24 hours at room temperature to form a gel composition.

### Example 2

| Ingredients | % w/w | Functions |
|---|---|---|
| Dapsone | 7.50 | Active |
| Diethylene glycol monoethylether | 20.00 | Solubilizing |
| (Transutol ® P, Gattefoose) | | Agent |
| Hydroxyethyl cellulose(HEC) | 6.00 | Gelling agent |
| (Natrosol ® 250 HHX Pharma | | |
| (Ashland)) | | |
| Glycerin | 3.00 | Emollient |
| Squalene | 2.50 | Emolient |
| Butylhydroxy toluene (BHT) | 0.05 | Preservative |
| Polysorbate 20 | 0.5 | Emollient |
| Demineralized Water | 60.45 | Aqueous phase |

### Manufacturing Process

1. Dapsone is solubilized into Diethylene glycol monoethyl ether.
2. Hydroxyethylcellulose is dispersed into glycerine and polysorbate 20 while stirring at approximately 350 rpm for 5 minutes. Add squalene and stir 10 minutes more.
3. Add BHT and water to dapsone solution while stirring ant 350 rpm for 20 minutes.
4. The phase with Dapsone is then added to the HEC phase under gentle stirring at 250 rpm and mixed for 2 hours.

### Example 3

| Ingredients | % w/w | Functions |
|---|---|---|
| Dapsone | 7.50 | Active |
| Diethylene glycol monoethylether | 20.00 | Solubilizing |
| (Transutol ® P, Gattefoose) | | Agent |
| Hydroxyethyl cellulose(HEC) | 5.00 | Gelling agent |
| (Natrosol ® 250 HHX Pharma | | |
| (Ashland)) | | |
| Glycerin | 3.00 | Emollient |
| Squalene | 2.00 | Emolient |
| Butylhydroxy toluene (BHT) | 0.05 | Preservative |
| Polysorbate 20 | 0.5 | Emollient |
| Demineralized Water | 61.95 | Aqueous phase |

### Manufacturing Process

1. Dapsone is solubilized into Diethylene glycol monoethyl ether.
2. Hydroxyethylcellulose is dispersed into glycerine and polysorbate 20 while stirring at approximately 350 rpm for 5 minutes. Add squalene and stir 10 minutes more.
3. Add BHT and water to dapsone solution while stirring ant 350 rpm for 20 minutes.
4. The phase with Dapsone is then added to the HEC phase under gentle stirring at 250 rpm and mixed for 2 hours.
5. The mixture is then left for 24 hours at room temperature to gel.

### Example 4

| Ingredients | % w/w | Functions |
|---|---|---|
| Dapsone | 7.50 | Active |
| Diethylene glycol monoethylether | 20.00 | Solubilizing |
| (Transutol ® P, Gattefoose) | | Agent |
| Hydroxyethyl cellulose(HEC) | 4.00 | Gelling agent |
| (Natrosol ® 250 HHX Pharma | | |
| (Ashland)) | | |
| Glycerin | 3.00 | Emollient |
| Squalene | 2.00 | Emolient |
| Butylhydroxy toluene (BHT) | 0.05 | Preservative |
| Polysorbate 20 | 0.5 | Emollient |
| Demineralized Water | 62.95 | Aqueous phase |

### Manufacturing Process

1. Dapsone is solubilized into Diethylene glycol monoethyl ether.
2. Hydroxyethylcellulose is dispersed into glycerine and polysorbate 20 while stirring at approximately 350 rpm for 5 minutes. Add squalene and stir 10 minutes more.
3. Add BHT and water to dapsone solution while stirring ant 350 rpm for 20 minutes.
4. The phase with Dapsone is then added to the HEC phase under gentle stirring at 250 rpm and mixed for 2 hours.

5. The mixture is then left for 24 hours at room temperature to gel.

### Example 5

| Ingredients | % w/w |
|---|---|
| Dapsone | 5.00 |
| Diethylene glycol monoethylether | 20.00 |
| (Transutol ® P, Gattefoose) | |
| Hydroxyethyl cellulose(HEC) | 2.00 |
| (Natrosol ® 250 HHX Pharma | |
| (Ashland)) | |
| Capryl-caproyl macrogol-8 | 10.00 |
| glyceride (Acconon ® MC8-2 | |
| EP/NF (Abitec)) | |
| Benzyl alcohol | 1.00 |
| Demineralized Water | 62.00 |

### Manufacturing Process

1. Dapsone is solubilized into Diethylene glycol monoethyl ether.
2. Add benzyl alcohol and water to dapsone solution while stirring at 350 rpm for 15 minutes and obtain phase with dapsone
3. Hydroxyethylcellulose is dispersed into capryl-caproyl macrogol-8 glyceride while stirring at approximately 350 rpm for at least 15 minutes and obtain HEC phase
4. The phase with dapsone is then added to the HEC phase under gentle stirring at 250 rpm and mixed for 3 hours.
6. The mixture is then left for 24 hours at room temperature to form a gel composition.

### Example 6

| Ingredients | % w/w |
|---|---|
| Dapsone | 7.50 |
| Diethylene glycol monoethylether | 30.00 |
| (Transutol ® P, Gattefoose) | |
| Hydroxyethyl cellulose(HEC) | 3.00 |
| (Natrosol ® 250 HHX Pharma | |
| (Ashland)) | |
| Capryl-caproyl macrogol-8 | 12.00 |
| glyceride (Acconon ® MC8-2 | |
| EP/NF (Abitec)) | |
| Benzyl alcohol | 1.00 |
| Demineralized Water | 46.50 |

### Manufacturing Process

1. Dapsone is solubilized into Diethylene glycol monoethyl ether.
2. Add benzyl alcohol and water to dapsone solution while stirring at 350 rpm for 15 minutes and obtain phase with dapsone
3. Hydroxyethylcellulose is dispersed into capryl-caproyl macrogol-8 glyceride while stirring at approximately 350 rpm for at least 15 minutes and obtain HEC phase
4. The phase with dapsone is then added to the HEC phase under gentle stirring at 250 rpm and mixed for 3 hours.
6. The mixture is then left for 24 hours at room temperature to form a gel composition.

### Example 7

| Ingredients | % w/w |
|---|---|
| Dapsone | 7.50 |
| Diethylene glycol monoethylether | 28.00 |
| (Transutol ® P, Gattefoose) | |
| Hydroxyethyl cellulose(HEC) | 3.00 |
| (Natrosol ® 250 HHX Pharma | |
| (Ashland)) | |
| Capryl-caproyl macrogol-8 | 14.00 |
| glyceride (Acconon ® MC8-2 | |
| EP/NF (Abitec)) | |
| Benzyl alcohol | 1.00 |
| Demineralized Water | 46.50 |

### Manufacturing Process

1. Dapsone is solubilized into Diethylene glycol monoethyl ether.
2. Add benzyl alcohol and water to dapsone solution while stirring at 350 rpm for 15 minutes and obtain phase with dapsone
3. Hydroxyethylcellulose is dispersed into capryl-caproyl macrogol-8 glyceride while stirring at approximately 350 rpm for at least 15 minutes and obtain HEC phase
4. The phase with dapsone is then added to the HEC phase under gentle stirring at 250 rpm and mixed for 3 hours.
6. The mixture is then left for 24 hours at room temperature to form a gel composition.

### Spreadability Test

Spreadability is expressed in terms of time in seconds taken by two slides to slip off from gel and placed in between the slides under the way of certain load. Smaller the time taken for separation of two slides better will be the spreadability of the formulations. Spreadability is a crucial characteristic of topical formulations and is responsible for correct dosage transfer to the target site and ease of application of the drug product which increases patient compliance as it would be known by one with ordinary skill in the art.

Spreadability is calculated using the following formula:

S=M×L/T

wherein, S=is the spreadability, M=is the weight (on the upper slide), L=is the length moved by the glass slide and T=represents the time taken to separate the slide completely from each other.

| | Spreadability |
|---|---|
| | (gm/cm2) |
| Example 1 | 12.1 |
| Example 2 | 11.5 |
| Example 3 | 11.7 |
| Example 4 | 12.3 |
| Example 5 | 10.7 |
| Example 6 | 11.3 |
| Example 7 | 10.3 |

### Appearence Test

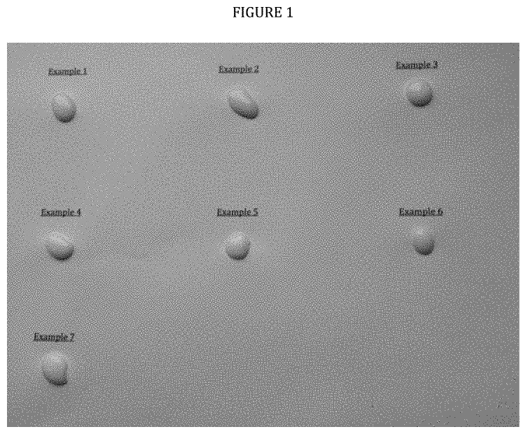

In another test, a sample of formulation examples 1, 2, 3, 4, 5, 6 and 7 were stored during 1 month in standard tubes at 20-25° C. at 60% relative humidity. It was found that there was no discoloration (yellowing) of the composition as seen in FIG. 1.

FIG. 1 demonstrates that there has been no color change (yellowing) with the formulations of the present invention (Examples 1-7) after 1 month at 20-25° C. at 60% relative humidity.

### Dapsone Crystal Particle Size Test

Samples were prepared by spreading the small amount of cream samples on microscope slides and the photos were taken by using Nikon Eclipse E200 Phase contrast inverted microscope with N is element software under the 10×CFI Plan acromate objectives and DS-FI3 camera. Hereunder, the size of the particles was measured and 100 dimension values were recorded in μm, and the mean particle size of the 100 dimension values was taken as the mean particle size.

TABLE 1
Mean particle size of dapsone

| | | | Manufac- | Manufac- | Manufac- |
|---|---|---|---|---|---|
| | Aczone | Aczone | turing | turing | turing |
| | 5% Gel | 7.5% Gel | Example 1 | Example 2 | Example 3 |
| Mean | 24.23 | 26.38 | 17.04 | 20.07 | 20.41 |
| Particle | | | | | |
| Size in μm | | | | | |

Table No:1 demonstrates the comparison between commercially sold Aczone 5% gel, 7,5% gel with example No.1 (EX1), example No.2(EX2) and example No.3 (EX3) prepared according to the present invention without an acrylamide derivative as a gelling agent (viscosity builder) and without a paraben. The data clearly demonstrate that Aczone 5% gel and Aczone 7.5% gel have larger crystals (which was taken as the average particle size of the crystals) compared to EX1, EX2and EX3. Larger crystals cause an increase to the mean particle size value of dapsone crystals. Thus, the mean particle size of dapsone crystals in the formulations according to the present invention is lower than the mean particle size in the formulations of the prior art Said reduction in particle size is beneficial for the application of the dapsone composition and increases the functional applicability of the gel, due to less friction (grittiness) and would increase the spreadability of the composition as known by one with ordinary skill in the art

1. A topical composition comprising: an effective amount of dapsone, at least one emollient, at least one solubilizing agent, and water, wherein the emollient is a glycerin based emollient present in an amount of between about 2% to about 20% by weight of the total composition and wherein the solubilizing agent is present in an amount between about 10 to about 40% by weight of the total composition.

2. The topical composition of claim 1, wherein the solubilizing agent and emollient have a weight ratio between of about 10:1 to about 1:1.

3. The topical composition of claim 1, wherein the of emollient(s) is present in a total amount of between about 5% to about 20% by weight of the total composition.

4. The topical composition of claim 1, wherein the emollient is selected from the group consisting of glycerin, caprylic/capric triglyceride macrogolglycerol ricinoleate, PEG glyceryl fatty acid esters such as PEG-8 glyceryl caprylate/caprate, PEG-4 glyceryl caprylate/caprate, PEG-32 glyceryl laurate, PEG-6 glyceryl mono oleate, PEG-6 glyceryl linoleate, glycerol triacetate, monoglycerides and acetylated monoglycerides, glycerol monodicocoate, glycerol monocaprylate, mono-and di-acetylated monoglycerides, polyglyceryl-3 dioleate, and combinations thereof.

5. The topical composition of claim 1, wherein the emollient is selected from the group consisting of glycerin, PEG-8 glyceryl caprylate/caprate, polyglyceryl-3 dioleate, PEG-4 glyceryl caprylate/caprate, PEG-32 glyceryl laurate, PEG-6 glyceryl mono oleate, PEG-6 glyceryl linoleate, macrogolglycerol ricinoleate, and combinations thereof.

6. The topical composition of claim 5, wherein the emollient is PEG-8 glyceryl caprylate/caprate, glycerin, and combinations thereof.

7. The topical composition of claim 1, wherein the solubilizing agent is selected from the group consisting of butylene glycol, polyethylene glycols (PEG), diethylene glycol monoethyl ether, tetrahydrofurfuryl PEG ether, N-methyl pyrrolidone, 2-Pyrrolidone, short chain alcohols, and combinations thereof.

8. The topical composition of claim 7, wherein the solubilizing agent is selected from the group consisting of diethylene glycol monoethyl ether, glycerol, N-methyl pyrrolidone, benzyl alcohol, and combinations thereof.

9. The topical composition of claim 8, wherein the solubilizing agent is diethylene glycol monoethyl ether.

10. The topical composition of claim 1, wherein the solubilizing agent is a combination of diethylene glycol monoethyl ether and a short chain alcohol.

11. The topical composition of claim 10, wherein the short chain alcohol is benzyl alcohol.

12. The topical composition of claim 1, wherein the composition further comprises at least one gelling agent.

13. The topical composition of claim 12, wherein the gelling agent is present in an amount of between about 1% to about 8% by weight of the total composition.

14. The topical composition of claim 13, wherein the amount of gelling agent is between about 3% to about 7% by weight of the total composition.

15. The topical composition according to claim 12, wherein the gelling agent is selected from the group consisting of carboxymethyl cellulose, ethylcellulose, hydroxyethyl cellulose, hydroxypropyl cellulose, methylcellulose, and combinations thereof.

16. The topical composition of claim 1, further comprising an emollient other than a glycerin based emollient.

17. The topical composition of claim 16, wherein the emollient other than glycerin based emollient is polysorbate, squalene, or combinations thereof.

18. The topical composition of claim 17, wherein the polysorbate is polysorbate 20.

19. The topical composition of claim 1, wherein the composition further comprises further comprising at least one preservative.

20. The topical composition of claim 19, wherein the preservative is butylhydroxy toluene or a short chain alcohol.

21. The topical composition of claim 1, wherein the composition further comprises a neutralizing agent.

22. The topical composition of claim 21, wherein the neutralizing agent is sodium hydroxide, triethanolamine, or combinations thereof.

23. The topical composition of claim 1, wherein the composition does not comprise a paraben.

24. The topical composition of claim 1, wherein the composition does not comprise an acrylamide based excipient.

25. The topical composition of claim 1, wherein the dapsone comprises crystals having a mean particle size of between about 10 to about 50 μm.

26. The topical composition of, wherein the mean particle size of dapsone crystals is between about 15 to about 30 μm.

27. A method for obtaining the topical composition according to claim 1, the method comprising the steps of mixing dapsone with a dispersion wherein the dispersion comprises a cellulose derivative dispersed into a glycerine based emollient.

28. The method of claim 27, wherein the method further comprises the steps of:
a. solubilizing dapsone with a solubilizing agent,
b. adding water and a preservative to the dapsone solution from step a),
c. dispersing a cellulose derivative into a glycerine based emollient with, a second or third emollient,
d. adding the cellulose phase from step c) to the dapsone solution obtained with the solubilizer, preservative and water from step b), and
e. leaving the mixture to gel.
